(19) United States
(12) Patent Application Publication
(10) Pub. No.: US 2006/0079841 A1
(43) Pub. Date: Apr. 13, 2006

(54) Rapid insufflation drug compartment

(75) Inventors: Henry J. Duff, Calgary (CA); Daniel E. Roach, Calgary (CA)

Correspondence Address: THOMPSON LAMBERT;SUITE 703D, CRYSTAL PARK TWO, 2121 CRYSTAL DRIVE, ARLINGTON, VA, 22202, US

(73) Assignee: University Technologies International Inc., Calgary (CA)

(21) Appl. No.: 10/959,061

(22) Filed: Oct. 7, 2004

(51) Int. Cl. A61M 5/00

(52) U.S. Cl. 604/116; 604/26; 600/439

(57) ABSTRACT

Subcutaneous injection of gas into a body causes insufflation of a cavity, which is followed by infusion of a fluid into the cavity. The fluid preferably carries an active agent, for example a dissolved gas or solute. The method may include iontophoresis of the cavity to enhance diffusion of the active agent into target tissue. Additional steps may be employed to monitor needle tip location to avoid causing damage to an organ by coming into contact with the needle tip or by injecting a gas into a blood vessel.

### BACKGROUND OF THE INVENTION

Various approaches have been used for drug delivery within animal bodies, including human bodies. Of these, perhaps oral delivery and direct injection are the most common. In addition, it has been proposed to administer drugs by injection into channels drilled into the body. Factors influencing the choice of drug delivery method include timing, such as the need for rapid diffusion of the drug into body tissue, and site specificity, such as when delivery of the drug to a specific organ or diseased area is required. This invention is directed towards an improved method of drug delivery. In addition, the method also provides for site specific cooling of body tissue.

### SUMMARY OF THE INVENTION

Therefore, it is an object of the invention to provide a method to enhance molecular and or thermal diffusion in tissue targets. The method is intended to be very rapid, very easy and flexible. According to an aspect of the invention, the method comprises subcutaneous injection of gas into a body to cause insufflation of a cavity followed by infusion of a fluid into the cavity. The fluid preferably carries an active agent, for example a dissolved gas or solute. The method may include iontophoresis of the cavity to enhance diffusion of the active agent into target tissue. Insufflation creates a closed conduit pneumatic flow path between an insufflation gas source and the cavity that allows solution to be injected into the cavity.

This method provides for enhanced thermal or molecular diffusion, and in a trauma situation may only take a few seconds for both injection steps. Injection of gases such as CO2 and NO cause vasodilation and increased permeability of microvasculature between cavity and skin or cavity and other tissue. Apparatus is also provided for insufflation followed by liquid injection, including a needle tip locator for avoiding organ damage from the needle tip of a catheter used to inject the gas and liquid.

These and other aspects of the invention are described in the detailed description of the invention and claimed in the claims that follow.

### BRIEF DESCRIPTION OF THE DRAWINGS

There will now be described preferred embodiments of the invention, with reference to the drawings, by way of illustration only and not with the intention of limiting the scope of the invention, in which like numerals denote like elements and in which:

FIG. 1 illustrates method steps according to an embodiment of the invention;

FIG. 2 shows an insufflation device including gas injector and components required for infusion of solution;

FIG. 3 shows a device for detecting needle tip proximity to blood flow using an ultrasonic source to detect the magnitude of Doppler-shifted reflected ultrasonic frequencies;

FIG. 4 shows a device for detecting needle tip proximity to blood flow using an ultrasonic reflectance profile;

FIGS. 5A and 5B show a device for detecting needle tip proximity to blood flow using dielectrics and capacitive sensing; and

FIG. 6 shows a device for detecting needle tip proximity to blood flow using reflective infra-red.

### DETAILED DESCRIPTION OF PREFERRED EMBODIMENTS

In this patent document, “comprising” means “including”. In addition, a reference to an element by the indefinite article “a” does not exclude the possibility that more than one of the element is present.

A harmless gas as used in this patent document is a gas that does not harm animal body tissue, as for example nitrous oxide, nitric oxide, carbon dioxide, oxygen and any of the noble gases. An interstitial space of an animal is a space between tissue of an animal, as for example a subcutaneous space or an intramuscular space, the insufflation of which does not significantly damage the tissue. The tissue may be muscle tissue or skin, and may be diseased tissue such as a tumour. A solution is a liquid carrying a solute. The solute may be an active agent. An active agent may be any compound having a beneficial effect on the animal, and includes any medical pharmaceutical and genetic material such as DNA and RNA. Examples of active agents include nutrients, blood products, preventive agents, for example vaccines, diagnostic agents, for example tracers of various types and imaging enhancers, therapeutic agents, for example drugs, peptides, and radiation, vaccine and virus vectors, and combinations of these classes. The active agent may include gas antidotes, biophysical modulators, for example paramagnetics, emitters, for example electromagnetic wave emitters, and imaging enhancers. The active agent may be carried as the solute in the electrolyte.

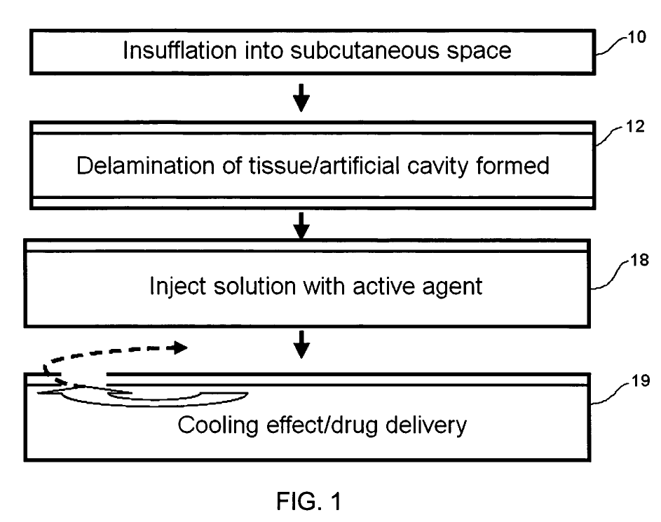

An animal, such as a human being, is treated by insufflation according to an embodiment of the invention by injecting a harmless gas, from a source of gas, into an interstitial space of the animal to create an artificial compartment within the animal and by infusing a solution into the artificial compartment. Basic method steps are illustrated in FIG. 1, in which insufflation 10 of gas into a subcutaneous space causes delamination 12 of tissue to form an artificial cavity. A fluid carrying an active agent is then injected 18 into the artificial cavity created by delamination 12. The active agent then diffuses 19 into surrounding tissue. In addition, dissolution or evaporation of gas present in the cavity cools the surrounding tissue.

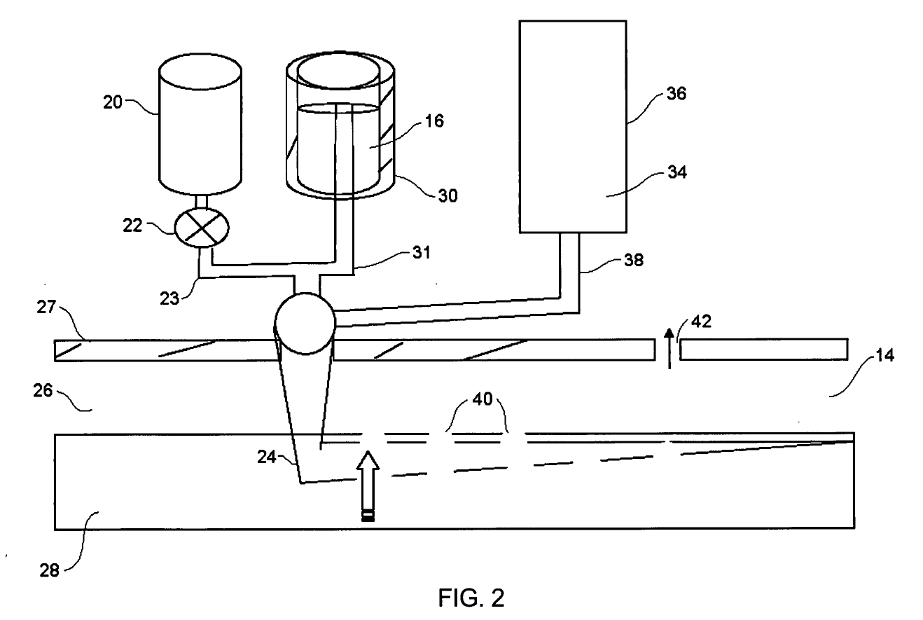

An apparatus for insufflation of tissue is shown in FIG. 2. In FIG. 2, gas injection in step 10 and 12 is provided from compressed gas cylinder 20 through gas regulator 22, flow line 23 and catheter 24, such as a conventional butterfly needle, into a subcutaneous or interstitial space 26, for example between skin 27 and tissue 28. The tissue surrounding the compartment 26 expands under gas pressure and the compartment forms a closed conduit pneumatic flow path with the source of injected gas 20 and the flow line 23 into which fluid may be injected. Fluid injection in step 18 is provided by cylinder 30, which may be designed to cool fluid 16 within the cylinder 30 by a Peltier effect cooling element with fan. Cylinder 30 is also connected by a flow line 31 to the catheter 24. Additional fluid 34 may be supplied by reservoir 36 connected to catheter 24 via flow line 38. The gas used for insufflation is typically either NO or CO2. Fluid 34 may be used to enhance the action of the active agent in the fluid 16, and may be for example a physiologic saline solution in the case when the fluid 16 is used to provide a cooling effect. The salt acts as a center for cavitation of the CO2 gas and thus causes CO2 gas to come out of solution. Dissolution of CO2 gas robs energy from the local environment, that is, the target tissue, thus cooling the target tissue.

Various designs of the catheter 24 may be used. For example, to enhance cooling, the catheter 24 could have multiple perforations 40 and a tapering structure to cause a venturi effect and thus potentially enhanced vaporization.

The solution 16 may be used to increase blood volume and maintain blood pressure. A preferred site for insufflation to increase blood volume is any interstitial space. Insufflation may be applied to an organ, such as for the delivery of pharmaceuticals or genetic material to the organ, where the organ has available delaminateable interstitial space for insufflation, and is not so vascular that there is a danger of the gas entering the blood stream. Insufflation of muscle may promote release of skeletal muscle stem cells into the circulation, and thus allow for regeneration of tissues requiring these stem cells after injury.

In particular, insufflation may be used as part of a process of cooling of tissue. In this embodiment, cooling may use the heat of dissolution, in which the gas coming out of solution absorbs heat from the surroundings, or the cooling effect of evaporation. In the example of infusion of carbonated solution 16, a needle is inserted into the cavity 14 to liberate the gas from the cavity 14 by creation of an exit wound 42. The release of the dissolved gas in the compartment 26 causes rapid local cooling of tissue surrounding the compartment. One example of cooling is cooling of the brain. Scalp tissues are insufflated by injection of gas under the scalp. Rapid release of gas by puncturing of the compartment causes local brain cooling, which may be advantageous for treating for example a stroke.

Iontophoresis may be applied to the electrolytic solution in the compartment. Iontophoresis is a system for promoting or accelerating transmittance of a drug molecule through a tissue barrier due to moving force of an electric field between an anode and cathode. [see Journal of Controlled Release, 18, 213-220 (1992); Advanced Drug Delivery Review, 9, 119 1992); and Pharmaceutical Research, 3, 318-326 (1986)]. Various means may be used to establish the electric field, such as by a first electrode extending into the compartment, and a second electrode applied to tissue adjacent the compartment.

The volume of the compartment 26 is dependent on the pressure used for insufflation. Large volumes of solute can be infused in a matter of minutes with for example a target pressure such as 1-5 psi. Preferred gases used for insufflation and dissolution include CO2, which causes local vasodilation, and NO, which causes intense vasodilation and increases permeability of the local vascular bed lining the artificial compartment 14. Other potential gases for use with an embodiment of the invention include noble gases, such as xenon and argon and pain relieving gases.

Injection of antibiotic into a compartment formed between fascial planes may be particularly suitable for diseases that follow fascial lines. As for example, insufflation interstitially between fascial planes at the site of flesh eating disease may be followed by injection of antibiotic into the resulting compartment and hence rapid delivery of the antibiotic to the site of the infection.

Example

Tissue in the anterior (front) leg of a dog was insufflated with a 2 second gas injection. The insufflation compartment was on the anterior aspect of the forelimb 2 cm before it joins the thorax. Then 100 cc of carbonated solution was injected into the insufflation compartment. An injectable thermometer was inserted in the center of a muscle 6 cm away. Baseline temperature of the compartment was 34.6 C. After CO2 insufflation, the temperature was 34.5 C. Cooled carbonated fluid was then injected into the cavity, causing the temperature to reduce to 34.4 C after three minutes. Gas was then allowed to flow from the exit wound for 1.5 minutes, and the temperature reduced to 32.4 C. After a further 5 minutes, the temperature was 32.6 C. Thus, insertion of the gas and cold carbonated solution had no significant effect on the temperature, but after releasing the CO2 gas to allow evaporation, the temperature dropped by 1.9 C in 3 minutes, and maintained this drop for as long as the CO2 was being injected into the cavity. Next, a dose of epinephrine (adrenalin) was injected into the cavity and intracavity inotophoresis was initiated. The heart rate of the dog increased from 80 to 87 beats per minute. This showed that insufflation can generate artificial compartments or cavities that can be brought into temperature equilibrium with surrounding tissues, and entry of drugs into the systemic circulation is feasible. Moreover, the cooling effect is apparent at a remote site 6 cm away from the insufflation with dense material, in this case the scapula of the subject dog, between the temperature sensor and the insufflation compartment. Thus, the cooling of an insufflation compartment has a cooling effect on tissues that are not in direct contact with the insufflation compartment.

Care should be taken to avoid placement of the needle in an organ or blood vessel. The length of the needle is chosen depending on the depth of the location of the compartment, and various known techniques may be used to prevent the needle from penetrating more than is desired, such as is taught for example in U.S. Pat. No. 6,656,160 issued Dec. 2, 2003, the content of which is hereby incorporated by reference. For insufflation of a tumour, the needle tip is placed into the tumour and a gas that is not absorbable by the tumour, such as nitrogen gas, is injected to insufflate the compartment. Care should be taken to avoid nitrogen entering the blood stream. If this cannot be avoided, due for example to the tumour being vascular, then nitrogen should be avoided.

Various needle tip locators, now known or later developed, for detecting needle tip proximity to blood vessels or organs may be employed. These may involve the measurement of ultrasonic frequencies, dielectric properties, resistivity of different tissues, or the reflection and absorption of infra-red.

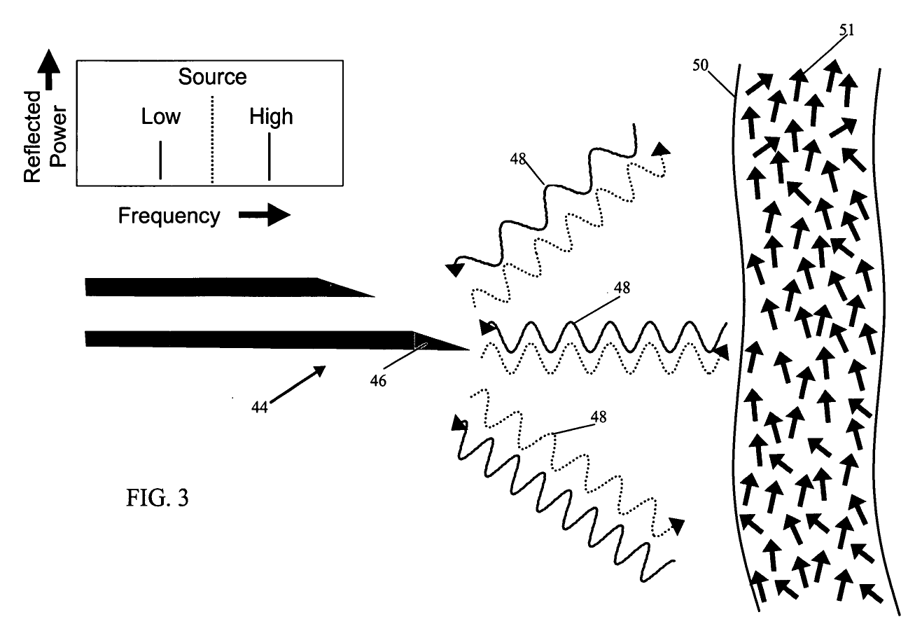

As shown in FIG. 3, a needle tip 44 is provided with a piezoelectric element 46 to detect “flow noises”, possibly in the ultrasonic domain. Doppler-shifted reflected signals 48 induced by blood flow in the vessel 50 are measured, with the strength of shifted return signals indicating proximity of the needle tip 44 to the flow, and thus the vessel 50.

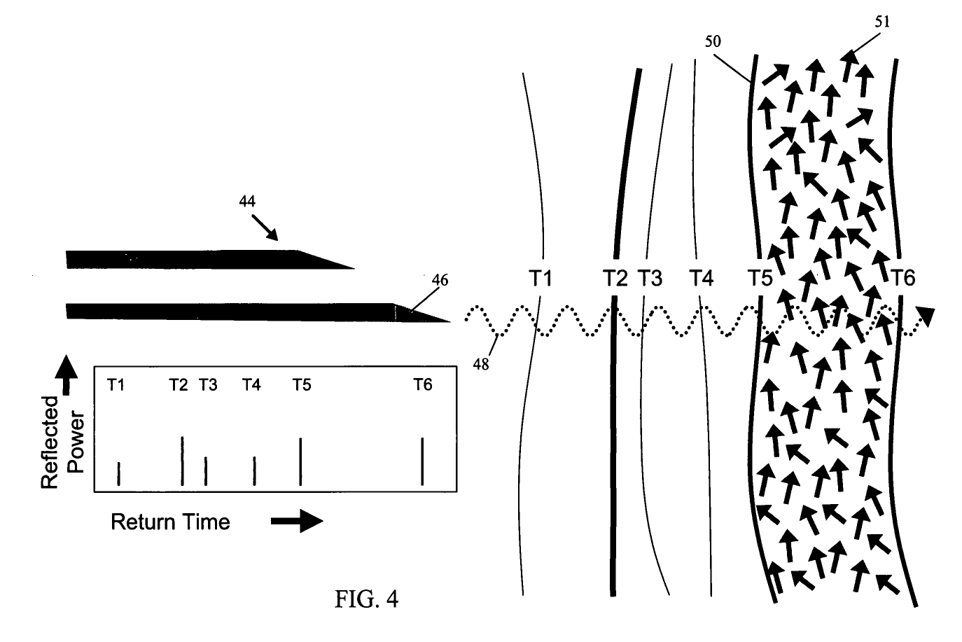

As shown in FIG. 4, a needle tip 44 is provided with a piezoelectric element 46 that emits an ultrasonic pulse 48 in the direction of the vessel 50, where T1 to T6 represent different tissue layers in the tissue environment surrounding the needle tip 44. By measuring the time and strength of the reflected return of the ultrasonic pulse 48, a one-dimensional view of the tissue environment surrounding the needle tip 44 is produced.

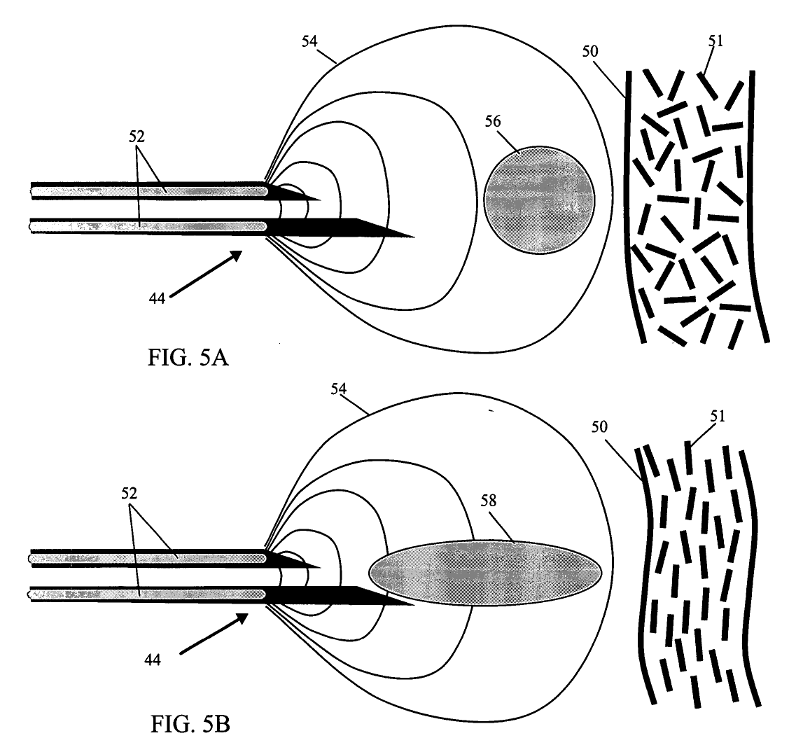

As shown in FIGS. 5A and 5B, a needle tip 44 is provided with a capacitive element 52 that produces an electric field 54. Different tissues have different dielectric properties. In blood vessel 50, the alignment of blood cells 51 during flow causes a spatial anisotropy 58 of the local dielectric conditions, thus providing a time-varying dielectric component. When the blood cells 51 are in a low flow environment, as shown in FIG. 5A, the flow results in an isotropic dielectric. This cyclic time-varying dielectric can be detected in several ways. The capacitive element 52 at the needle tip 44 will detect the time-varying capacitance caused by the flow-induced time-varying dielectric strength. A simple ratiometric capacitance circuit can be used to detect the magnitude of the changing capacitance. Similarly, micro impedance plethysmography may be used to detect the change in impedance resulting from the flow-induced time-varying change in dielectric strength.

Alternatively, tissue resistivity may be used to detect needle tip proximity to blood flow. Different tissues have different resistivities. Therefore, tip electrical resistance may be measured to indicate the type of tissue adjacent to the needle tip.

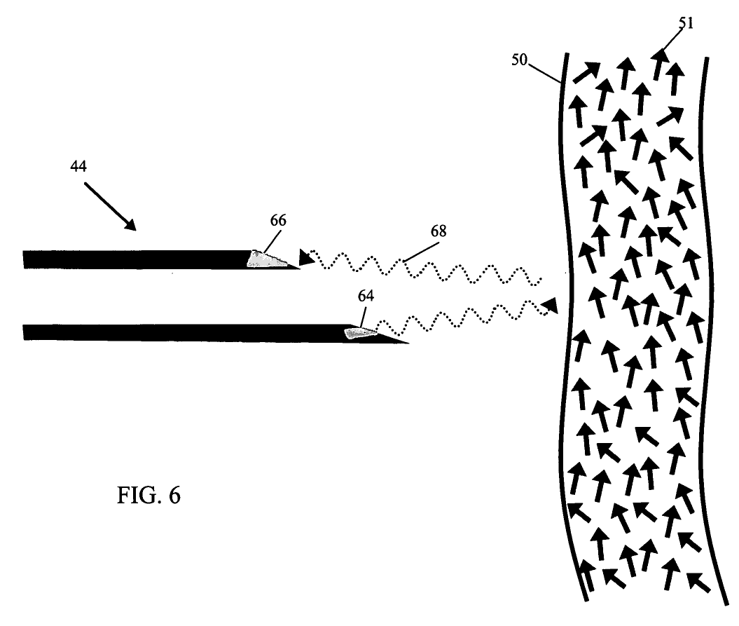

As shown in FIG. 6, a needle tip 44 is provided with a confocal infra-red transmitter 64 and receiver 66 pair. The transmitter 64 emits an infra-red signal 68 in the direction of the vessel 50. Flow dynamics within the vessel 50 will produce cyclic changes in the absorption/reflectivity properties of the blood cells 51. The reflected infra-red signal 68 is modulated by anisotropy created by the alignment of blood cells 51 during blood flow. Proximity to the blood flow within the vessel 50 will be indicated by pulsatility strength of the reflected infra-red signal 68.

Immaterial modifications may be made to the embodiments described here without departing from the invention.

What is claimed is:

1. A method of treating an animal, the method comprising the steps of:
injecting a harmless gas into an interstitial space of the animal to create an artificial compartment within the animal; and
infusing a solution into the artificial compartment.

2. The method of claim 1 in which the solution carries an active agent.

3. The method of claim 2 in which the solution is electrolytic and further comprising applying an electric field to the electrolytic solution to enhance transmittance of the active agent into tissue.

4. The method of claim 1 in which the solution contains a dissolved gas.

5. The method of claim 4 further comprising releasing gas from the artificial compartment to cool tissue surrounding the artificial compartment.

6. The method of claim 5 in which the solution contains a nucleating agent.

7. The method of claim 1 in which the artificial compartment is created by delamination of tissue under gas pressure.

8. The method of claim 7 in which the artificial compartment is created between fascial planes.

9. A method of treating an animal having a tumour, the method comprising the steps of:
injecting a harmless gas into the tumour to create an artificial compartment within the tumour; and
infusing a solution into the artificial compartment.

10. The method of claim 9 in which the solution carries an anti-tumour agent.

11. The method of claim 10 further comprising applying an electric field to the solution to enhance transmittance of the active agent into tissue.

12. The method of claim 1 further comprising the steps of:
inserting a needle through tissue into the interstitial space, the needle having a tip;
monitoring needle tip location during insertion of the needle through the tissue; and
injecting the harmless gas through the needle.

13. The method of claim 12 in which the needle tip comprises a piezoelectric element.

14. The method of claim 12 in which monitoring needle tip location comprises the steps of:
directing an ultrasonic source at the tissue;
measuring the strength of Doppler-shifted return signals induced by blood flow in a vessel; and
stopping insertion of the needle based on the strength of the Doppler-shifted return signals.

15. The method of claim 12 in which monitoring needle tip location comprises the steps of:
directing an ultrasonic pulse at the tissue;
measuring the time and strength of the ultrasonic pulse reflected from the tissue; and
producing a one-dimensional view of the tissue surrounding the needle tip.

16. The method of claim 12, in which the needle tip comprises a capacitive element that produces an electric field.

17. The method of claim 16, in which the capacitive element detects time-varying capacitance caused by a flow-induced time-varying dielectric strength,

18. The method of claim 16 in which micro impedance plethysmography is used to detect a change in impedance resulting from a flow-induced time-varying change in dielectric strength.

19. The method of claim 12 in which the needle tip comprises a sensor for measuring electrical resistance.

20. The method of claim 19 in which electrical resistance is measured to indicate the type of tissue adjacent to the needle.

21. The method of claim 12 in which the needle tip comprises a con-focal infra-red transmitter and receiver pair.

22. The method of claim 21 monitoring needle tip location comprises the steps of:
emitting an infra-red signal in the direction of the tissue;
measuring pulsatility strength of the infra-red signal reflected from the tissue; and
stopping forward movement of the needle based on a pre-determine value of the pulsatility strength.

23. The method of claim 22 in which the tissue is blood within a blood vessel.

24. The method of claim 12 in which monitoring needle tip location is used to avoid damage to an organ from the needle tip.

25. The method of claim 12 in which monitoring needle tip location is used to avoid injecting a gas into a blood vessel.

26. An apparatus for insufflation of tissue, the apparatus comprising:
a catheter having a needle tip;
a first flow line connected to the cathether, the first flow line being connected to a source of pressurized gas and having a gas flow regulator;
a second flow line connected to the catheter, the second flow line being connected to a liquid source and having a liquid flow regulator; and
a needle tip locator for locating the needle tip of the catheter within animal tissue to prevent damage to an organ.

27. The apparatus of claim 26 in which the needle tip locator comprises a piezoelectric element

28. The apparatus of claim 26 in which the needle tip locator comprises a capacitive element that produces an electric field.

29. The apparatus of claim 26 in which the needle tip locator comprises a sensor for measuring electrical resistance.

30. The apparatus of claim 26 in which the needle tip locator comprises a con-focal infra-red transmitter-receiver pair.

31. The apparatus of claim 26 in which the organ is a blood vessel.

32. A closed conduit pneumatic flow path, comprising:
a source of pressurized gas;
a compartment in animal tissue formed by expansion and delamination of tissue by the pressurized gas;
a flow line leading from the source of pressurized gas to a catheter having a needle tip located within the compartment; and
a needle tip locator for locating the needle tip within the compartment.

33. The closed conduit pneumatic flow path of claim 32 further comprising a solution within the compartment that has been injected from the needle tip.
